(19) United States
(12) Patent Application Publication
(10) Pub. No.: US 2021/0107928 A1
(43) Pub. Date: Apr. 15, 2021

(54) SPHINGOSINE-1-PHOSPHATE ANALOG AND SYNTHESIS METHOD THEREFOR

(71) Applicant: SEJONG BIOMED CO., LTD., Gyeonggi-do (KR)

(72) Inventors: Yong Tae KIM, Sejong (KR); In Suk HONG, Sejong (KR); Eun Jin KIM, Gyeonggi-do (KR); Chan Hee CHON, Gyeonggi-do (KR); Seong Hwan CHO, Gyeonggi-do (KR)

(21) Appl. No.: 16/965,056

(22) PCT Filed: Jan. 28, 2019
(86) PCT No.: PCT/KR2019/001159

§ 371 Date: Jul. 27, 2020

(30) Foreign Application Priority Data
Jan. 29, 2018 (KR) 10-2018-0010516

(51) Int. Cl. C07F 9/09; C07C 239/20; G01N 33/544; G01N 33/543

(52) U.S. Cl. CPC C07F 9/091; G01N 33/54353; G01N 33/544; C07C 239/20

(57) ABSTRACT

The present specification relates to a sphingosine-1-phosphate (S1P) analogue and a method of synthesizing the same. A novel S1P analogue disclosed by the present specification is highly water-soluble and highly stable due to an alkoxyamine group thereof. Thus, the novel S1P analogue is suitable for use in manufacturing an immunodiagnostic kit.

### TECHNICAL FIELD

Embodiments disclosed by the present specification relate to a sphingosine-1-phosphate (S1P) analogue and a method of synthesizing the same, and more particularly to a S1P-alkoxyamine compound, which is a S1P analogue, and a method of synthesizing the same.

### BACKGROUND ART

Sphingosine-1-phosphate (S1P) is a type of sphingolipid among lysolipids and is also referred to as D-erythro-sphingosine-1-phosphate. S1P is produced by the phosphorylation of sphingosine by sphingosine kinase. In particular, S1P is accumulated a lot in platelets, and is known to be released into the blood by the activation of platelets.

Researchers at the National Institute of Allergy and Infectious Diseases (NIAID) under the National Institutes of Health published a paper showing that S1P in the blood plays an important role in maintaining a balance between bone formation and destruction (Sphingosine-1-phosphate mobilizes osteoclast precursors and regulates bone homeostasis, Nature, Feb. 8, 2009). This suggests that S1P may be a new therapeutic target for the diagnosis and treatment of bone-related degenerative diseases such as osteoporosis and rheumatoid arthritis.

The Asan Foundation applied for and registered a patent which relates to a marker composition for predicting the risk of fracture or osteoporosis including S1P on the basis that S1P appears at a high level regardless of bone density in individuals with fractures (KR101486368B1).

Currently, S1P is a marker that can diagnose not only bone-related diseases, but also various pathophysiological conditions, particularly cancer, inflammation, angiogenesis, heart disease, asthma, and autoimmune disease, and furthermore, research results have shown that S1P can be used as a therapeutic target. Therefore, in order to develop a kit for diagnosing various diseases, there is a need to develop an economical and efficient synthetic process for S1P or S1P analogues.

Conventionally, ECHELON manufactures and sells S1P and S1P analogues, i.e., S1P-fluorescein and S1P-TAMRA. In addition, the patent US20070281320A1 of Lpath, Inc. discloses a S1P analogue having a sulfhydryl group, a carboxylic acid group, a cyano group, an ester, a hydroxy group, an alkene, an alkyne, an acid chloride group, or a halogen atom, at sn-1 of S1P. Thiolated-S1P of the formula below, which is disclosed as a major S1P analogue in the patent specification of Lpath, Inc., has low water solubility, and thus can be manipulated by applying an organic solvent or heat, and is easily oxidized in air to thereby form a disulfide bond, and thus due to these chemical properties, it is not easy to purify, store, and use thiolated-S1P. Accordingly, it is not easy to use thiolated-S1P in an immunodiagnostic kit using S1P.

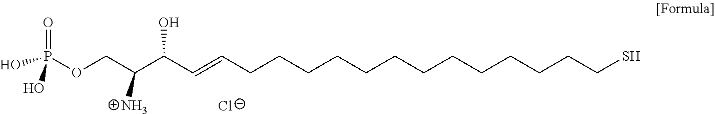

In addition, manufacturing immunodiagnostic kits involves fixing a S1P analogue to a plate or support member, but conventional thiolated-S1P, which is disclosed in the patent specification of Lpath, Inc., cannot be directly coupled to a plate or support member. Thus, in order to fix thiolated-S1P to a plate or a support member, a separate linker needs to be used. For example, to fix thiolated-S1P to a protein molecule as a support member, i.e., keyhole limpet hemocyanin (KLH) or bovine serum albumin (BSA), a separate linker, i.e., iodoacetamide (IOA) or succinimidyl-4-(N-maleimidomethyl) cyclohexane-1-carboxylate (SMCC) needs to be used. Accordingly, there is a disadvantage that a process for manufacturing an immunodiagnostic kit is complicated.

Therefore, under the circumstance in which a S1P analogue compound, which is easy to purify, store, and use, and a method of mass-synthesizing the compound need to be developed, the inventors of the present specification had studied S1P analogues useful for manufacturing an immunodiagnostic kit, and confirmed S1P analogues having high water solubility and high stability against oxidization and capable of being attached to a plate of an immunodiagnostic kit even without a separate linker, and a method of synthesizing the same, thus completing the present application.

### DESCRIPTION OF EMBODIMENTS

### Technical Problem

An object to be achieved by the content disclosed by the present application is to provide a novel sphingosine-1-phosphate (S1P) analogue.

Another object to be achieved by the content disclosed by the present application is to provide a method of synthesizing a novel S1P analogue.

Still another object to be achieved by the content disclosed by the present application is to provide a use of a novel S1P analogue and/or a method of synthesizing the same.

### Technical Solution

According to an aspect of the present application, in order to achieve the aforementioned objects, there is provided with a novel sphingosine-1-phosphate (S1P) analogue. The S1P analogue is a S1P analogue compound selected from compounds of Formula 1 below.

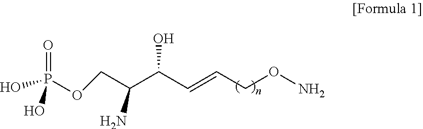

[Wherein the n is selected from an integer of 2 to 13 in the Formula 1.]

According to another aspect of the present application, in order to achieve the aforementioned objects, there is provided with a method of synthesizing a novel S1P analogue. The synthesis method is a method of preparing a compound of Formula 1 below, including: a) preparing a compound of Formula 4 below by changing a hydroxyl group contained in a compound of Formula 3 below into another functional group having less reactivity than the hydroxyl group; b) preparing a compound of Formula 5 below from the compound of Formula 4 below using an amino alcohol precursor; c) preparing a compound of Formula 6 below from the compound of Formula 5 below using a reductant; d) preparing a compound of Formula 7 below from the compound of Formula 6 below using an anhydride; e) preparing a compound of Formula 8 below from the compound of Formula 7 below by changing the functional group introduced in process a) above into a hydroxyl group; f) preparing a compound of Formula 9 below by changing the hydroxyl group contained in the compound of Formula 8 below into a better leaving group than the hydroxyl group; g) preparing a compound of Formula 10 below from the compound of Formula 9 below by changing the functional group introduced in process f) above; and h) preparing a compound of Formula 2 below from the compound of Formula 10 below by reacting with tetrahydrofuran (THF) and hydrochloric acid. The S1P analogue synthesized by the synthesis method has a structure of Formula 1 below.

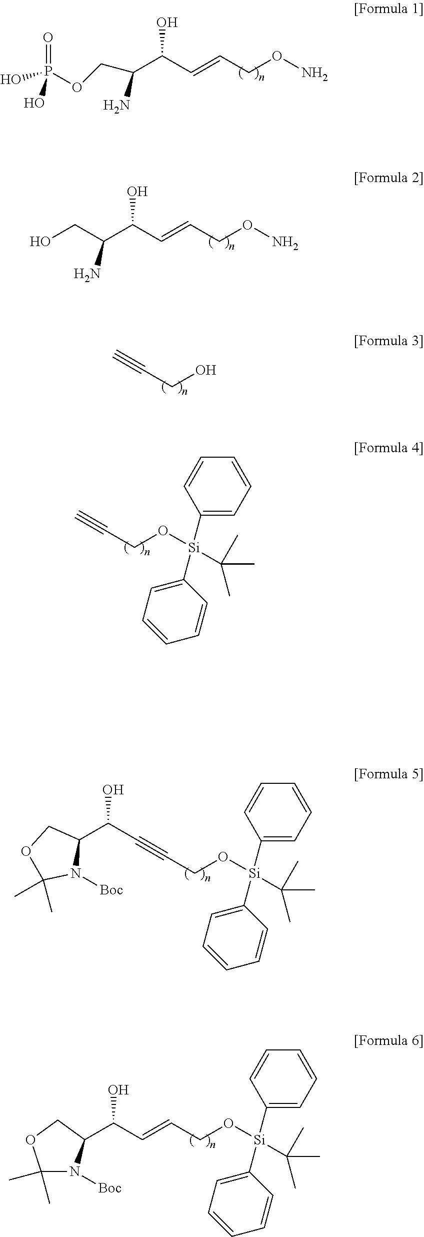

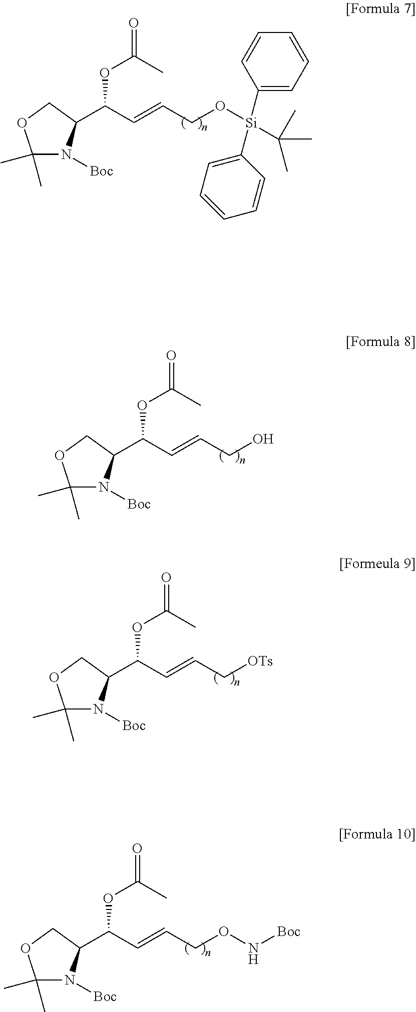

[Wherein the n is selected from an integer of 2 to 13 in the Formula 1 to 10.]

According to still another aspect of the present application, in order to achieve the aforementioned objects, there is provided with an immunodiagnostic kit including: a plate; and a sphingosine-1-phosphate (S1P) analogue fixed to the plate and selected from the compounds of Formula 1.

### Advantageous Effects

The technology disclosed by the present specification has the following effects.

First, a novel sphingosine-1-phosphate (S1P) analogue can be provided. Furthermore, a S1P analogue having a higher reactivity and higher water solubility can be provided.

Second, a method of synthesizing a novel S1P analogue can be provided. Furthermore, a method of synthesizing a S1P analogue, which is more economical and has a high yield, can be provided.

Third, an economically synthesized S1P analogue can be more easily applied to an immunodiagnostic kit, and thus can contribute to immunodiagnosis of S1P-related pathophysiological conditions.

### BRIEF DESCRIPTION OF DRAWINGS

FIG. 1 illustrates a sphingosine-1-phosphate (S1P) analogue according to an embodiment.

FIG. 2 illustrates a S1P analogue having a specific chemical structure according to an embodiment.

FIG. 3 illustrates process a) of a method of synthesizing a S1P analogue, according to an embodiment.

FIG. 4 illustrates process b) of a method of synthesizing a S1P analogue, according to an embodiment.

FIG. 5 illustrates process c) of a method of synthesizing a S1P analogue, according to an embodiment.

FIG. 6 illustrates process d) of a method of synthesizing a S1P analogue, according to an embodiment.

FIG. 7 illustrates process e) of a method of synthesizing a S1P analogue, according to an embodiment.

FIG. 8 illustrates process f) of a method of synthesizing a S1P analogue, according to an embodiment.

FIG. 9 illustrates process g) of a method of synthesizing a S1P analogue, according to an embodiment.

FIG. 10 illustrates process h) of a method of synthesizing a S1P analogue, according to an embodiment.

FIG. 11 illustrates process i) of a method of synthesizing a S1P analogue, according to an embodiment.

FIG. 12 illustrates process j) of a method of synthesizing a S1P analogue, according to an embodiment.

FIG. 13 illustrates process k) of a method of synthesizing a S1P analogue, according to an embodiment.

FIG. 14 illustrates the overall process of synthesizing a S1P analogue having a specific chemical structure according to an embodiment.

FIGS. 15 and 16 illustrate immunodiagnostic kits including a S1P analogue according to an embodiment.

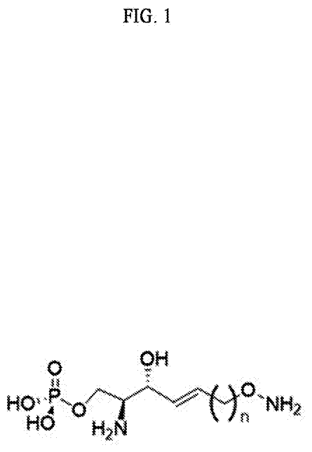

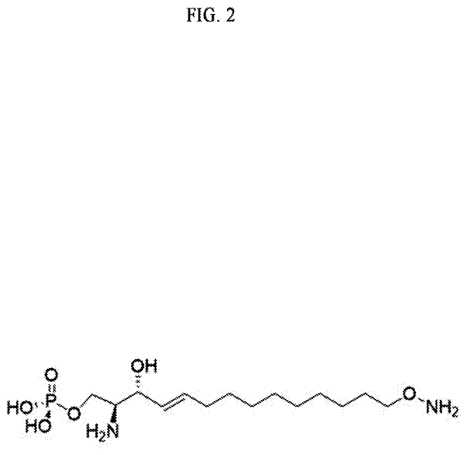

### BEST MODE

Definitions of representative terms used in the present specification are as follows.

The term “analogue” refers to a compound having a chemical structure similar to a specific organic compound, for example, some of the atoms constituting a molecule are substituted with another element or deleted, or another element is added thereto.

The term “biomarker” is a specific biochemical in the body with certain molecular properties that makes it useful to measure the progression of a disease or the effectiveness of treatment. For example, S1P is a biomarker for cancer, inflammation, angiogenesis, heart disease, asthma, autoimmunity, or osteoporosis.

The term “immunodiagnosis” refers to diagnosis according to the test results obtained by immunological techniques, and means confirmation of the presence or absence and degree of an antigen in a biological sample. Methods known in the art may be used for the immunodiagnosis, and may be, for example, enzyme-linked immunosorbent assay (ELISA) and point-of-care testing (POCT), but the present specification is not limited thereto. For example, precipitation method, agglutination method, immunofluorescence staining, enzyme immunoassay, radioimmunoassay, and chemiluminescence immunoassay may be used, but the present specification is not limited thereto.

The term “epitope” refers to a specific portion of an antigen that allows the immune system, such as antibodies, B cells, T cells, and the like, to identify the antigen.

The term “separation” or “purification” means removing or diluting one or more other compounds from an active compound. Compound components that can be removed or diluted during the separation or purification process include chemical reaction products, unreacted chemicals, proteins, carbohydrates, lipids, and unbound molecules.

The term “about” means an amount, level, value, number, frequency, percentage, size, weight, or length that varies by about 30%, about 25%, about 20%, about 25%, about 10%, about 9%, about 8%, about 7%, about 6%, about 5%, about 4%, about 3%, about 2%, or about 1% with respect to a reference amount, level, value, number, frequency, percentage, size, weight, or length.

In addition to these terms, other terms are defined elsewhere in the specification, as necessary. Unless explicitly defined otherwise herein, terms, as used herein and used in the art, will have general meanings recognized in the art.

Hereinafter, the content disclosed by the present specification will be described in detail.

Signal Transducing Bioactive Lipids

Lipids and derivatives thereof are now recognized as important targets for medical research, not as mere structural elements in cell membranes or as a source of energy for β-oxidation, glycolysis, or other metabolic processes. In particular, certain bioactive lipids function as signal transduction mediators important in animal and human diseases. Although most of the lipids of the plasma membrane merely play an structural role, a small proportion of them are involved in relaying extracellular stimuli into cells. Lipid signal transduction pathways are activated by a variety of extracellular stimuli factors, including growth factors and inflammatory cytokines, and regulate cell fate decisions such as apoptosis, differentiation and proliferation.

Lysolipids

Lysolipids are low molecular weight lipids that contain a polar head and a single hydrocarbon backbone. With regard to the polar head at sn-3, the hydrocarbon chain may be located at sn-2 and/or sn-1 position(s). These lipids represent signal transduction bioactive lipids, and their biological and medical importance is highlighted in that the lipid signal-transducing molecules can be targeted for achieving a healing, diagnostic/prevention or research purposes. A specific example of a medically important lysolipid is S1P (sphingoid skeleton). Other lysolipids include LPA (glycerol skeleton), sphingosine, lysophosphatidylcholine (LPC), sphingosylphosphorylcholine (lysospingomyelin), ceramide, ceramide-1-phosphate, sphinganine (dihydrosphingosine), dihydrosphingosine-1-phosphate, and N-acetyl-ceramide-1-phosphate.

Sphingolipids

Sphingolipids are a class of lipids containing a backbone of the sphingoid bases and aggregates of aliphatic amino alcohols. Sphingolipids are the primary structural components of cell membranes that also act as cell signaling and regulatory molecules. The structural backbones of S1P, dihydro-S1P (DHS1P), and sphingosylphosphorylcholine (SPC) are based on sphingosine derived from sphingomyelin. Sphingolipid signal transduction mediators, ceramide (CER), sphingosine (SPH), and sphingosine-1-phosphate (S1P) have been studied most extensively, and their role in cardiovascular system, angiogenesis and tumor biology has recently been recognized.

Sphingosine-1-Phosphate (S1P)

Extensive expression of cell surface S1P receptors allows S1P to influence a wide spectrum of cellular responses, including proliferation, adhesion, contraction, motility, morphogenesis, differentiation, and survival. This response spectrum depends on overlapping or unique expression patterns of S1P receptors in cellular and tissue systems. Modulation of various cellular processes involving S1P has particular effects on neuronal signaling, vascular tone, wound healing, immune cell trafficking, reproduction, and cardiovascular function. Altering the endogenous levels of S1P in these systems can have adverse effects leading to several pathophysiological conditions including cancer, inflammation, angiogenesis, heart disease, asthma, and autoimmune diseases. Thus, S1P can be a biomarker capable of diagnosing the risk of developing several pathophysiological conditions, particularly cancer, inflammation, angiogenesis, heart disease, asthma, autoimmunity, or bone-related diseases.

In order to manufacture an immunodiagnostic kit using S1P, which is highly available as a biomarker, it is necessary to fix (attach) S1P to a plate constituting the kit or a support member attached to the plate. The plate is an element constituting the immunodiagnostic kit, and may provide a space where a biomarker, a buffer solution, and the like are contained. The plate may be made of a polymer material such as polystyrene, polypropylene, polycarbonate, or nylon, but the present specification is not limited thereto. The plate may consist of various synthetic polymers such as nitrocellulose, cellulose, cellulose acetate, and polyethylene, but the present specification is not limited thereto. The support member may be another element constituting the immunodiagnostic kit. The support member may directly or indirectly contact the plate to provide a role of the support so that the biomarker S1P can be fixed. The support member may consist of glass, polysaccharides, polyacrylamide, polystyrene, polyvinyl alcohol, silicone, or protein molecules, but the present specification is not limited thereto.

S1P is a chemical structure having a hydrocarbon chain at sn-1 and a polar head at sn-3. In order to perform immunodiagnosis using S1P, the polar head, which is a major part of the S1P epitope, must be exposed. Therefore, the S1P immunodiagnostic kit requires immobilization on the plate using the sn-2 or sn-1 portion excluding the polar head region of the S1P analogue. Immobilization to the plate using the hydrocarbon chain of the sn-2 or sn-1 portion of S1P is not easy in terms of reactivity. Therefore, in order to use S1P as a biomarker, a method of separately synthesizing a S1P analogue that is easy to immobilize on a plate and immobilizing the corresponding analogue to a plate of an immunodiagnostic kit should be used.

Alkoxyamines can form chemical bonds through chemical reactions with specific functional groups. For example, the alkoxyamine is highly reactive with an epoxy group, and thus can form a chemical bond. For example, the alkoxyamine has high reactivity with a molecule having a carbonyl group, and thus can form a chemical bond to form a very stable oxime compound. The molecule having a carbonyl group may be a molecule including aldehydes, ketones, carboxylic acids and derivatives thereof such as esters and amides, and ketenes, but the present specification is not limited thereto. Therefore, the S1P-alkoxyamine compound may be provided in the manufacture of an immunodiagnostic kit.

According to an embodiment disclosed by the present specification, there is provided a method for synthesizing a novel S1P analogue.

According to an embodiment disclosed by the present specification, there is provided a method of synthesizing a S1P analogue, particularly S1P-alkoxyamine, from 1-alkyne-n-ol (HCC(CH₂)ₙ—OH).

For example, a method of synthesizing a S1P analogue, particularly S1P-alkoxyamine, from 1-alkyne-n-ol (HCC(CH₂)ₙ—OH), is a method of synthesizing S1P-alkoxyamine of Formula 1 below, which is a S1P analogue, from a compound of Formula 3 below, which is 1-alkyne-n-ol (HCC(CH₂)ₙ—OH), including: A method for producing an analogue of sphingosine-1-phosphate (S1P) of a formula 1 below, comprising:

a) Preparing a compound of Formula 4 below by changing a hydroxyl group contained in a compound of Formula 3 below into another functional group having less reactivity than the hydroxyl group;

b) Preparing a compound of Formula 5 below from a compound of Formula 4 below by using an amino alcohol precursor;

c) Preparing a compound of Formula 6 below from a compound of Formula 5 below by using a reductant;

d) preparing a compound of Formula 7 below from a compound of Formula 6 below by using an anhydride;

e) Preparing a compound of Formula 8 below from a compound of Formula 7 below by changing a functional group introduced in the a) process into a hydroxyl group;

f) Preparing a compound of Formula 9 below by changing the hydroxyl group contained in a compound of Formula 8 below into a better leaving group than the hydroxyl group;

g) Preparing a compound of Formula 10 below from a compound of formula 9 below by changing into the functional group introduced in the f) process; and

h) Preparing a compound of Formula 2 below from a compound of Formula 10 below by reacting with tetrahydrofuran (THF) and hydrochloric acid;

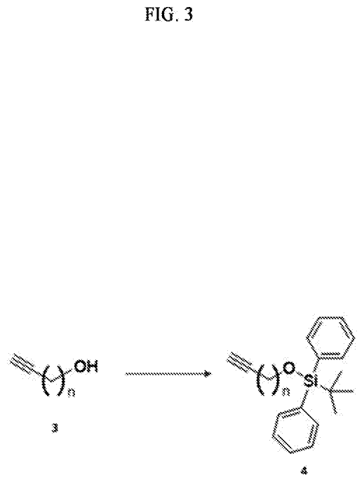

The process a) is a process of preparing a compound of Formula 4 below by changing a hydroxyl group included in a compound of Formula 3 below into another functional group having less reactivity than the hydroxyl group (FIG. 3).

Hydrogen of the hydroxyl group may be removed from the compound of Formula 3 through the process a).

Through the process a), the hydrogen of the hydroxyl group contained in the compound of Formula 3 may be substituted with another functional group having less reactivity than the hydroxyl group to thereby prepare the compound of Formula 4.

Hydrogen of the hydroxyl group contained in the compound of Formula 3 may be substituted with a hydroxyl-group-protecting group to thereby prepare the compound of Formula 4.

The process a) is to protect an oxygen atom at the sn-1 position of the hydrocarbon chain during the processes as described below.

The hydroxyl-group-protecting group may have lower reactivity than a hydroxyl group with respect to the processes as described below, and as will be described in detail in process e), the hydroxyl-group-protecting group may be substituted again with a hydrogen atom by a compound used in process e).

Thus, the hydroxyl group located at the sn-1 position of the hydrocarbon chain of the compound of Formula 3 may be protected in processes b) to process d). Other functional groups that are less reactive than the hydroxyl group include at least one selected from a group consisting of tert-butyldimethylsilyl ether, tert-butyldiphenylsilyl ether, and triisopropylsilyl ether. Other functional groups having less reactivity than the hydroxyl group may be used in an amount of 1 equivalent to 4 equivalents, but the present specification is not limited thereto.

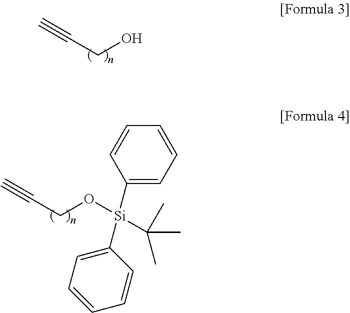

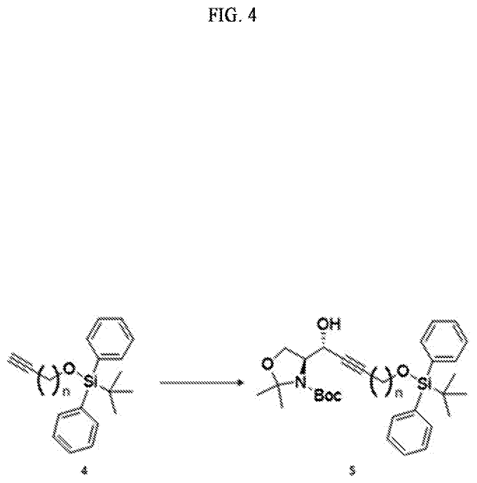

The process b) is a process of preparing a compound of Formula 5 below from the compound of Formula 4 using an amino alcohol precursor (FIG. 4).

Through the process b), hydrogen linked to a carbon triple bond may be removed from the compound of Formula 4.

Hydrogen linked to a carbon triple bond in the compound of Formula 4 may be substituted with an amino alcohol precursor to thereby prepare the compound of Formula 5.

The process b) is to provide an amino alcohol precursor to the sn-3 position of the hydrocarbon chain during the processes as described below.

The process b) is to provide an amino alcohol by the processes as described below, and as will be described in detail in process h), the amino alcohol precursor may be changed into an amino alcohol by a compound used in process h).

Thus, hydrogen linked to a carbon triple bond located at the sn-3 position of the hydrocarbon chain of the compound of Formula 4 may provide an amino alcohol precursor in process c) to process g).

The amino alcohol precursor may be a compound having a structure in which N of the amino alcohol is forward and a structure in which a hydroxyl group is backward.

The amino alcohol precursor may be Garner's aldehyde of Formula 13 below.

In the process b), the amino alcohol precursor may be used with organolithium compound.

The organolithium compound includes at least one selected from a group consisting of n-butyllithium, sec-butyllithium, and tert-butyllithium. The organolithium compound may be used in an amount of 1 equivalent to 4 equivalents, but the present specification is not limited thereto.

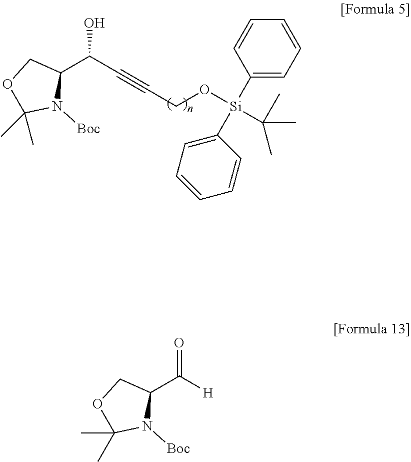

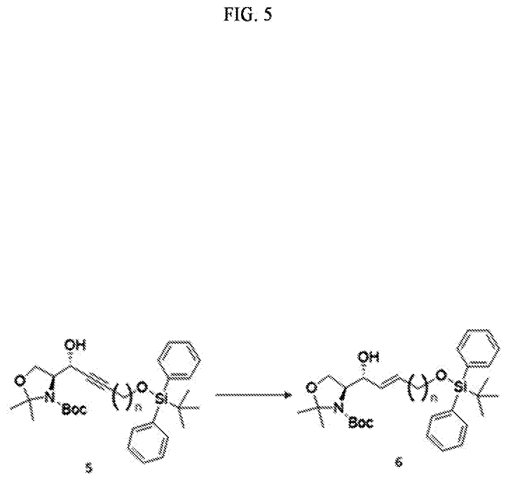

The process c) is preparing a compound of Formula 6 below from the compound of Formula 5 by using reductant (FIG. 5).

Through the process c), a carbon triple bond of the compound of Formula 5 may be changed into a carbon double bond.

Hydrogen may be introduced into the carbon triple bond in the compound of Formula 5 by the process c), thereby preparing the compound of Formula 6 having a carbon double bond.

In the compound of Formula 5, the carbon triple bond may be reduced to thereby prepare the compound of Formula 6 having a carbon double bond.

A reductant may be used to reduce the carbon triple bond.

As the reductant, a reagent for reducing a carbon triple bond to a carbon double bond may be used, herein, the reagent maintains the structure in which N of the amino alcohol is forward and a hydroxyl group is backward at the sn-3 position, respectively.

The process c) is to provide a carbon double bond form to a hydrocarbon chain during the processes as described below.

Red-Al (sodium bis(2-methoxyethoxy)aluminumhydride; NaAlH₂(OCH₂CH₂OCH₃)₂) of a compound of Formula 14 below may be used as the reductant.

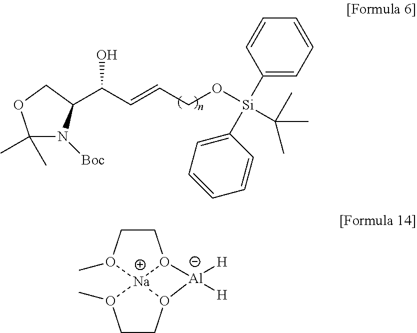

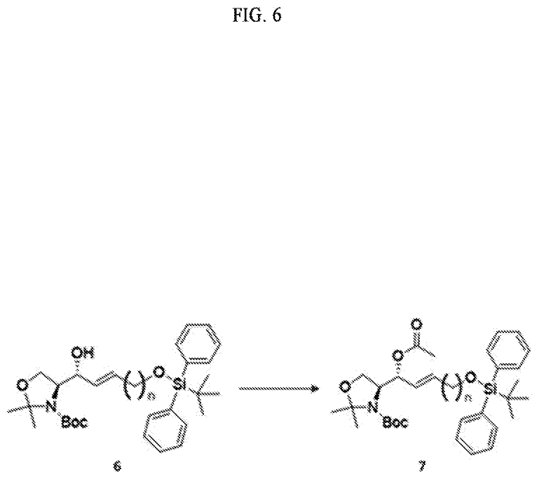

The process d) is preparing a compound of Formula 7 below from the compound of Formula 6 by using an anhydride (FIG. 6).

Through the process d), hydrogen may be removed from a hydroxyl group bound to C3 from the compound of Formula 6.

Hydrogen of the hydroxyl group bound to C3 in the compound of Formula 6 may be acetylated by the process d).

Through the process d), hydrogen of the hydroxyl group bound to C3 in the compound of formula 6 may be substituted with an acetyl group to thereby prepare the compound of Formula 7.

Hydrogen of the hydroxyl group in the compound of Formula 6 may be acetylated by an anhydride provided along with pyridine to thereby prepare the compound of Formula 7. The process d) may have lower reactivity than the hydroxyl group at C3 for the processes as described below, and as will be described in detail in process h), the acetyl group may be substituted again with a hydrogen atom by the compound used in process h).

Thus, the hydroxyl group linked to C3 of the compound of Formula 6 may be protected in processes e) to g).

In the process d), the anhydride includes at least one selected from the group consisting of acetic anhydride and acetyl halide (CH₃—CO—X) of Formula 15 below. The anhydride compound may be used in an amount of 10 equivalents to 20 equivalents, but the present specification is not limited thereto.

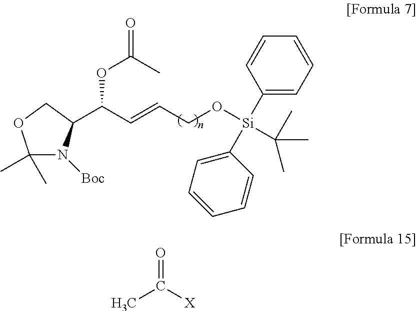

[Wherein the X is selected from a halogen atom in the Formula 15.]

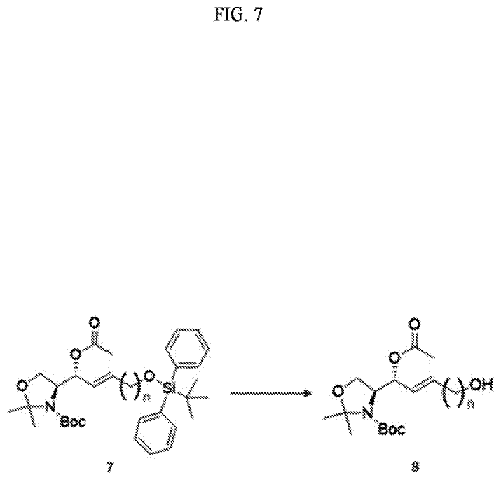

The process e) is a process of preparing a compound of Formula 8 below from the compound of Formula 7 by changing the functional group introduced in the process a) into a hydroxyl group (FIG. 7).

The hydroxyl-group-protecting group may be removed from the compound of Formula 7 by the process e).

Through the process e), the compound of Formula 8 may be prepared from the compound of Formula 7 by substituting the hydroxyl-group-protecting group with a hydrogen atom.

The hydroxyl-group-protecting group in the compound of Formula 7 may be substituted with a hydrogen atom, thereby preparing the compound of Formula 8 having a hydroxyl group at the sn-1 of a hydrocarbon chain.

The process e) is to provide a hydroxyl group for process f), which will be described below.

The hydroxyl group may have higher reactivity than the hydroxyl-group-protecting group for the processes as described below, and as will be described in detail in process f), the hydroxyl group may be substituted again by a compound used in process f).

Thus, the hydroxyl-group-protecting group located at the sn-1 position of the hydrocarbon chain of the compound of Formula 7 is substituted with the hydroxyl group located at the sn-1 position of the compound of Formula 8, and is provided for the process described below.

In the process e), a compound including at least one selected from a group consisting of KF, CsF, HF, and n-Bu₄NF may be used. The compound may be used in an amount of 5 equivalents to 10 equivalents, but the present specification is not limited thereto.

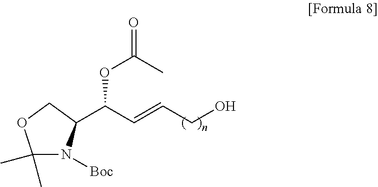

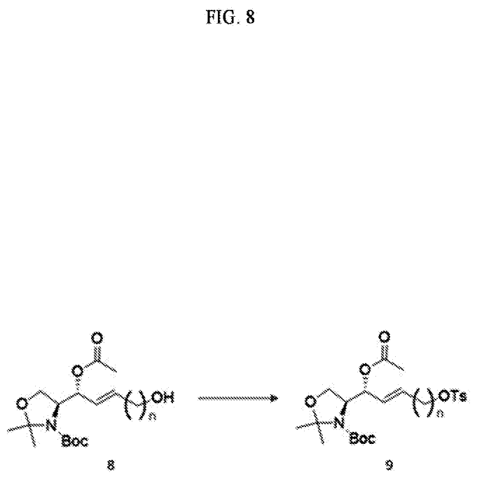

The process f) is a process of preparing a compound of Formula 9 below by changing the hydroxyl group contained in the compound of Formula 8 into a better leaving group than the hydroxyl group (FIG. 8).

The hydroxyl group may be removed from the compound of Formula 8 by the process f).

Through the process f), the compound of Formula 9 may be prepared from the compound of Formula 8 by substituting the hydroxyl group with another functional group that functions better as a leaving group.

The better leaving group is a functional group that performs a nucleophilic reaction better than a hydroxyl group.

The hydroxyl group in the compound of Formula 8 may be substituted with a better leaving group to thereby prepare the compound of Formula 9.

The process f) is to provide a functional group that performs a nucleophilic reaction better than the hydroxyl group located at the sn-1 position of the hydrocarbon chain during the processes as described below.

The better leaving group may have properties as a better leaving group than the hydroxyl group for the processes as described below, and as will be described in detail in process g), the better leaving group may be removed by a compound used in process g).

Thus, the hydroxyl group located at the sn-1 position of the hydrocarbon chain of the compound of Formula 8 is substituted with better leaving group, which is located at the sn-1 position of the compound of Formula 9, and is provided for the process described below.

The leaving group that is better than the hydroxyl group of the process f) may include at least one selected from a group consisting of p-toluenesulfonate, methanesulfonate, p-nitrobenzoate, p-nitrobenzenesulfonate, p-bromobenzenesulfonate, and trifluoromethanesulfonate. The functional group may be used in an amount of 10 equivalents to 30 equivalents, but the present specification is not limited thereto.

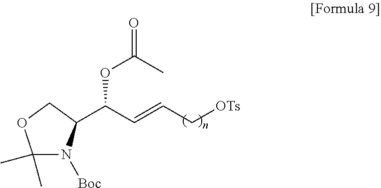

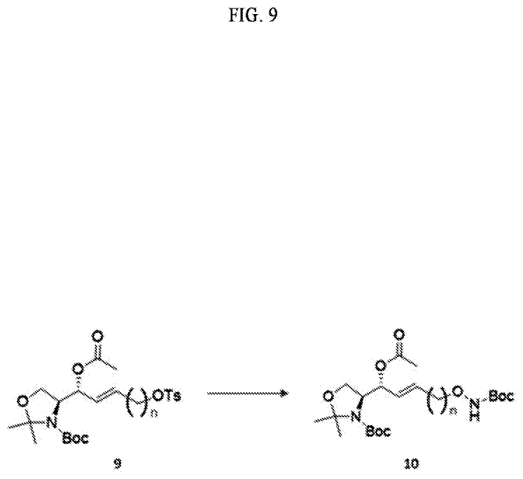

The process g) is a process of preparing a compound of Formula 10 below from the compound of Formula 9 by changing the functional group introduced in the process f) (FIG. 9).

A compound that modifies the functional group introduced in the process g) may be an N-Boc-hydroxylamine compound of Formula 16 below.

The better leaving group in the compound of Formula 9 may be removed by the process g).

Through the process g), the better leaving group in the compound of Formula 9 may cause a nucleophilic reaction with an N-Boc-hydroxylamine compound.

The N-Boc-hydroxylamine may be used in combination with 1,8-Diazabicyclo[5.4.0]undec-7-ene (DBU).

The DBU may provide a function of activating a hydroxyl group of N-Boc-hydroxylamine.

The better leaving group located at the sn-1 position in the compound of Formula 9 may be substituted with O—N-Boc to thereby prepare the compound of Formula 10.

The process g) is to provide O—NH-Boc at the sn-1 position of the hydrocarbon chain during the processes as described below.

The O—NH-Boc serves to provide a precursor of an amine, more specifically, a precursor of an alkoxyamine, for the processes as described below, and as will be described in detail in process k), the O—NH-Boc may be substituted with an alkoxyamine by a compound used in process k).

Thus, the better leaving group located at the sn-1 position of the hydrocarbon chain in the compound of Formula 9 is substituted with O—NH-Boc located at the sn-1 position of Formula 10, and is provided for the process described below.

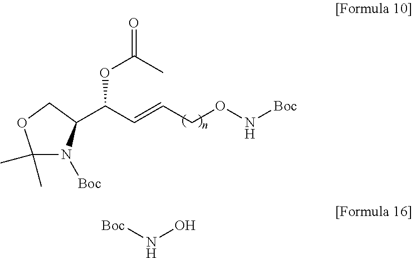

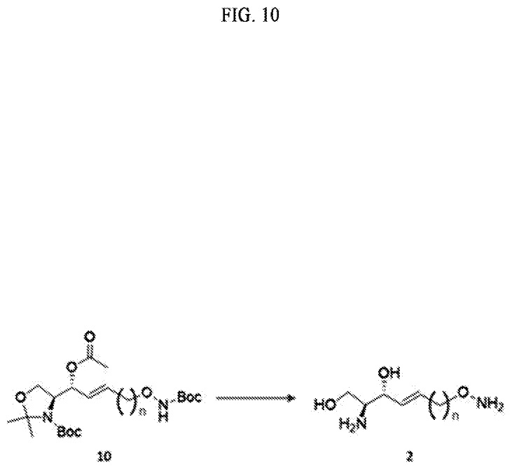

The process h) is a process of preparing a compound of Formula 2 below from the compound of Formula 10 by reacting with tetrahydrofuran (THF) and hydrochloric acid (FIG. 10).

The amino alcohol precursor in the compound of Formula 10 may be changed by the process h).

Through the process h), the amino alcohol precursor in the compound of Formula 10 may be changed into an amino alcohol to thereby prepare the compound of Formula 11.

Through the process h), an acetonide in the compound of Formula 10 may be changed into an amino alcohol to thereby prepare the compound of Formula 11.

Through the process h), Boc may be removed from the compound of Formula 10.

Through the process h), Boc in the compound of Formula 10 may be substituted with a hydrogen atom to thereby prepare the compound of Formula 11.

The amino alcohol precursor in the compound of Formula 10 may be reduced to prepare the compound of Formula 11 having an amino alcohol at the sn-3 position thereof.

The process h) may provide an amino alcohol at the sn-3 position during the processes as described below, and as will be described in detail in process i), some of the hydrogen atoms of the amino alcohol may be substituted with Boc in process i).

Thus, the amino alcohol precursor located at the sn-3 position of the hydrocarbon chain of the compound of Formula 10 is substituted with an amino alcohol, which is located at the sn-3 position of Formula 11, and provided for the process as described below.

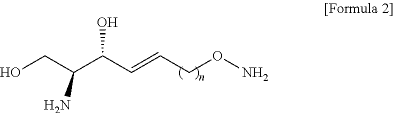

[Wherein the n is selected from an integer of 2 to 13, in the Formula 2 to Formula 10.]

According to another embodiment disclosed by the present specification, another method of synthesizing a S1P analogue, particularly S1P-alkoxyamine is further provided.

For example, as the method of synthesizing a S1P analogue, i.e., the S1P-alkoxyamine of Formula 1, a method of preparing a sphingosine-1-phosphate analogue further including, in addition to processes a) to h) as described above,

i) preparing a compound of Formula 11 below by changing the amine group contained in the compound of Formula 2 into another functional group that has less reactivity than the amine group is further provided.

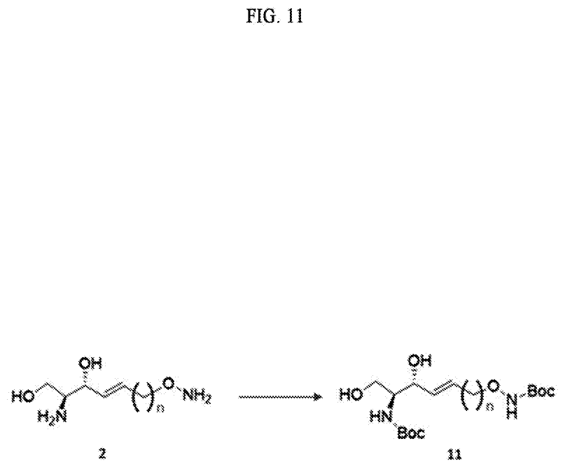

The process i) is a process of preparing the compound of Formula 11 below by changing the amine group contained in the compound of Formula 2 below to another functional group having less reactivity than the amine group (FIG. 11).

Through the process i), one hydrogen atom of the amine group linked to C2 and the amine group located at the sn-1 position is removed from the compound of Formula 2.

Through the process i), the compound of Formula 11 may be prepared from the compound of Formula 2 by substituting one hydrogen atom of the amine group linked to C2 and the amine group located at the sn-1 position with Boc.

When one hydrogen atom of the amine groups is substituted with Boc compared to the amine groups, reactivity may be reduced compared to the amine groups.

The Boc may function as an amine-group-protecting group.

The compound of Formula 11 may be prepared from the compound of Formula 2 by substituting one hydrogen atom of the amine group linked to C2 and the amine group located at the sn-1 position with another functional group having less reactivity than the hydrogen atom.

One hydrogen atom of the amine group linked to C2 and the amine group located at the sn-1 position in the compound of Formula 2 may be substituted with Boc to thereby prepare the compound of Formula 11.

The process i) is to protect the amine group linked to C2 and the amine group located at the sn-1 position during the processes as described below.

The amine-group-protecting group may have lower reactivity than the amine group for the processes as described below, and as will be described in detail in process k), the amine-group-protecting group may be substituted again with a hydrogen atom by a compound used in process k).

Thus, the amine group linked to C2 of the hydrocarbon chain and the amine group located at the sn-1 position of the compound of Formula 2 may be protected in process j).

Other functional groups having less reactivity than the amine group in the process i) may include at least one selected from a group consisting of di-tert-butyl dicarbonate (Boc₂O), 9-fluorenylmethyl carbamate, tert-butyl carbamate, benzyl carbamate, acetamide, trifluoroacetamide, phthalimide, benzylamine, triphenylmethylamine, benzylideneamine, and p-toluenesulfonamide. Other functional groups having less reactivity than the amine group may be used in an amount of 2 equivalents to 5 equivalents, but the present specification is not limited thereto.

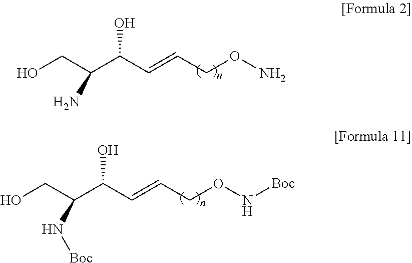

According to another embodiment disclosed by the present specification, another method for synthesizing a S1P analogue, particularly S1P-alkoxyamine is further provided.

For example, as the method of synthesizing a S1P analogue, i.e., the S1P-alkoxyamine of Formula 1, a method of preparing sphingosine-1-phosphate analogue further including, in addition to processes a) to i) as described above,

j) preparing a compound of Formula 12 below from the compound of Formula 11 using a phosphonate ester is further provided.

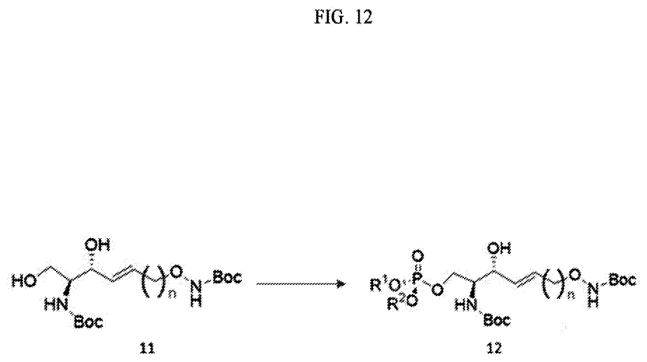

The process j) is a process of preparing a compound of Formula 12 below from the compound of Formula 11 using a phosphonate ester (FIG. 12).

Through the process j), hydrogen of the hydroxyl group located at the sn-3 position may be removed from the compound of Formula 11.

Through the process j), hydrogen of the hydroxyl group linked to C1 may be removed from the compound of Formula 11.

Through the process j), the compound of Formula 12 may be prepared from the compound of Formula 11 by substituting the hydrogen of the hydroxyl group located at the sn-3 position with PO(OR″²)₂.

The hydrogen of the hydroxyl group located at the sn-3 position in the compound of Formula 11 may be substituted with PO(OR″²)₂ to thereby prepare the compound of Formula 12.

The process j) is to provide phosphonate ester at the sn-3 position of the hydrocarbon chain during the processes as described below.

The phosphonate ester may provide a phosphate for the processes described below, and as will be described in detail in process k), the residue of the phosphonate ester may be substituted with a hydrogen atom.

In the process j), N-methylimidazole (NMI) of Formula 17 below may be used in combination with the phosphonate ester.

The NMI, which is a base, may be used to activate a hydroxyl group linked to C1.

The phosphonate ester may include at least one selected from a group consisting of phosphonate esters (R³PO(OR^{1,2})₂) of Formula 18 below. The phosphonate ester compound may be used in 2 equivalents to 20 equivalents, but the present specification is not limited thereto.

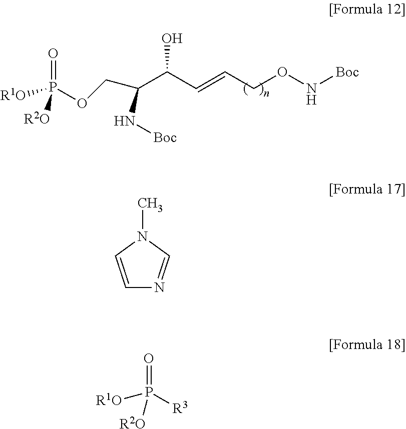

[In the Formula 12 and 18, R³ is selected from Halogen atom, HSO₄ or p-toluenesulfonate except for fluorine, and is selected from H or alkyl group having C1 to C7.]

According to another embodiment disclosed by the present specification, another method of synthesizing a S1P analogue, particularly S1P-alkoxyamine, is further provided.

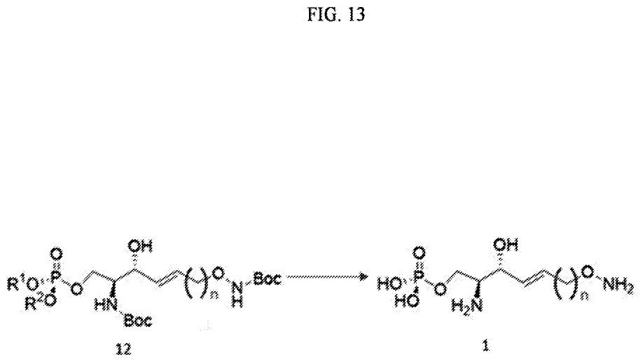

For example, as the method of synthesizing a S1P analogue, i.e., the S1P-alkoxyamine of Formula 1, a method of preparing a sphingosine-1-phosphate analogue further including, in addition to processes a) to j) as described above, k) preparing a compound of Formula 1 below by changing an O-acyl group contained in the compound of Formula 12 with a hydrogen atom is further provided. The process k) is a process of preparing a compound of Formula 1 below by changing the O-acyl group contained in the compound of Formula 12 into a hydrogen atom (FIG. 13).

Through the process k), the O-acyl group contained in the compound of Formula 12 may be removed.

Through the process k), OR¹, OR², Boc bound to N linked to C2, and Boc at the sn-1 position of the phosphonate ester included in the compound of Formula 12 may be substituted with hydrogen atoms to thereby prepare the compound of Formula 13.

The compound of Formula 13 may be prepared from the compound of Formula 12 by substituting the O-acyl groups with hydrogen atoms.

The O-acyl groups are OR¹, OR², Boc bound to N linked to C2, and Boc at the sn-1 position of the phosphonate ester.

The OR′ and Ole of the phosphonate ester are substituted with hydrogen atoms so that a phosphate is provided at the sn-3 position of the compound of Formula 13.

Boc bound to N linked to C2 and Boc at the sn-1 position are substituted with hydrogen atoms so that an amine and an alkoxyamine group are provided at C2 and the sn-1 position, respectively, of the compound of Formula 13.

Thus, the O-acyl groups of Formula 12 are used to provide a phosphate at the sn-3 position of the compound of Formula 13, an amine group at C2 thereof, and an alkoxyamine group at the sn-1 position thereof.

For the reaction that changes the O-acyl groups into hydrogen atoms, a compound including at least one selected from a group consisting of trimethylsilyl bromide (Me₃SiBr), tribromoborane (BBr₃), hydrobromate (HBr), and dimethylformamide may be used. The compound that changes the O-acyl groups into hydrogen atoms may be used in an amount of 5 equivalents to 15 equivalents.

The compound that changes the O-acyl groups into hydrogen atoms may be used together with water.

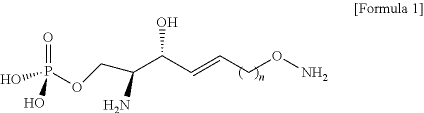

[Wherein the n is selected from an integer of 2 to 13, in the Formula 1, 2, 11 and 12.]

Common technical details for each process can be understood with reference to conventional organic synthetic methods known in the art.

According to another embodiment disclosed by the present specification, a method of synthesizing, from 1-alkyne-n-ol (HCC(CH₂)ₙ—OH), a sphingosine analogue, particularly sphingosine-alkoxyamine, is provided.

For example, the method of synthesizing, from 1-alkyne-n-ol (HCC(CH₂)ₙ—OH), a sphingosine analogue, particularly sphingosine-alkoxyamine, is a method of synthesizing sphingosine-alkoxyamine of Formula 2, which is a sphingosine analogue, from the compound of Formula 3, i.e., 1-alkyne-n-ol (HCC(CH₂)ₙ—OH),

A method for producing an analogue of sphingosine of Formula 2 above, comprising:

a) Preparing the compound of Formula 4 by changing a hydroxyl group in the compound of Formula 3 into another functional group having less reactivity than the hydroxyl group;

b) Preparing the compound of Formula 5 from the compound of Formula 4 by using an amino alcohol precursor;

c) Preparing the compound of Formula 6 from the compound of Formula 5 by using a reductant;

d) Preparing the compound of Formula 7 from the compound of Formula 6 by using an anhydride;

e) Preparing the compound of Formula 8 from the compound of Formula 7 by changing the functional group introduced in the process a) into a hydroxyl group;

f) Preparing the compound of Formula 9 by changing a hydroxyl group contained in the compound of Formula 8 into a better leaving group than the hydroxyl group;

g) Preparing the compound of Formula 10 from the compound of Formula 9 by changing the functional group introduced in the process f); and

h) Preparing the compound of Formula 2 from the compound of Formula 10 by reacting with tetrahydrofuran (THF) and hydrochloric acid.

Detailed descriptions of processes a) to h) are the same as those described above for each process.

According to another embodiment disclosed by the present specification, a novel sphingosine-1-phosphate (S1P) analogue compound is provided.

According to an embodiment disclosed by the present specification, a sphingosine-1-phosphate (S1P) analogue, particularly S1P-alkoxyamine of Formula 1 below, is provided.

According to another embodiment disclosed by the present specification, a sphingosine analogue, particularly sphingosine-alkoxyamine of Formula 2 below, is provided.

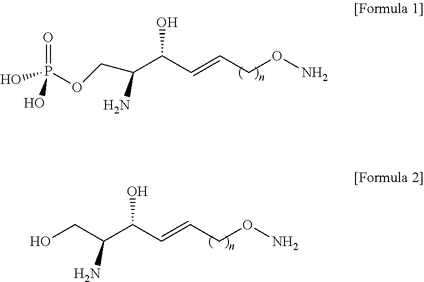

[Wherein the n is selected from an integer of 2 to 13, in the Formula 1 and Formula 2.]

The novel S1P analogue disclosed in the present specification has an alkoxyamine group at sn-1. The alkoxyamine group is hydrophilic and has a chemical property of being a functional group with high oxidation degree. Accordingly, S1P-alkoxyamine, which is a novel S1P analogue disclosed in the present specification, is water-soluble, and barely undergoes additional oxidation in air, and thus is highly stable against oxidation in air. In addition, the alkoxyamine group may form a bond with a specific functional group that specifically or selectively performs a chemical reaction. The specific functional group includes, but is not limited to, an epoxy group and a carbonyl group. The carbonyl group may be included in an aldehyde, a ketone, a carboxylic acid and derivatives thereof such as an ester and an amide, and a ketene, but the present specification is not limited thereto. Accordingly, S1P-alkoxyamine, which is a novel S1P analogue disclosed in the present specification, may form a bond with a specific functional group that specifically or selectively performs a chemical reaction.

Therefore, according to the present specification, a S1P analogue with enhanced water solubility and enhanced stability against oxidation in air is provided. In addition, a S1P analogue capable of forming a bond with a specific functional group that specifically or selectively performs a chemical reaction with an alkoxyamine is provided.

According to another embodiment disclosed by the present specification, an immunodiagnostic kit including the S1P analogue as a use of a novel S1P analogue compound and/or a method of synthesizing the same is provided.

According to an embodiment disclosed by the present specification, an immunodiagnostic kit including a S1P analogue, particularly S1P-alkoxyamine, is provided.

For example, an immunodiagnostic kit is provided, the kit comprises:

a plate; and a sphingosine-1-phosphate analogue that is selected from a compound of Formula 1 below, which is capable of bounding the plate.

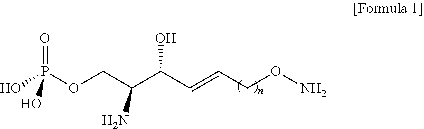

[Wherein the n is selected from an integer of 2 to 13, in the Formula 1.]

The plate may be coated with S1P. The plate may be made of a polymer material such as polystyrene, polypropylene, polycarbonate, or nylon, but the present specification is not limited thereto. The plate may consist of various synthetic polymers such as nitro cellulose, cellulose, cellulose acetate, and polyethylene, but the present specification is not limited thereto.

The plate may have a functional group capable of reacting with the alkoxyamine group of the S1P analogue. The functional group may be an epoxy group or a carbonyl group, but the present specification is not limited thereto. The carbonyl group may be included in an aldehyde, a ketone, a carboxylic acid and derivatives thereof such as an ester and an amide, and a ketene, but the present specification is not limited thereto.

The plate may directly and/or indirectly immobilize a S1P analogue. The plate includes a functional group, and the functional group and the alkoxyamine group of the S1P analogue may form a bond through a chemical reaction. Thus, the S1P analogue may be immobilized directly and/or indirectly on a plate containing a functional group. For example, the S1P-alkoxyamine compound may be immobilized directly and/or indirectly on an epoxidized plate. Alternatively, for example, the S1P-alkoxyamine compound may be immobilized directly and/or indirectly on a plate containing a carbonyl group.

According to another embodiment disclosed by the present specification, another immunodiagnostic kit using a S1P analogue, particularly S1P-alkoxyamine, is further provided.

For example, an immunodiagnostic kit is provided, the kit comprises:

a plate; and sphingosine-1-phosphate that is selected from a compound of Formula 1 below, which is capable of bounding the plate, the immunodiagnostic kit further comprising a support member.

The support member may serve to mediate the plate and S1P analogue. The support member may have a structure in contact with the plate. The support member may have a structure in contact with the S1P analogue. The support member may be a structure in direct and/or indirect contact with the plate. The support member may be a structure in direct and/or indirect contact with the S1P analogue.

The support member may consist of glass, polysaccharides, polyacrylamide, polystyrene, polyvinyl alcohol, silicone, or protein molecules, but the present specification is not limited thereto. The support member may preferably be polyacrylamide or a protein molecule. The support member may more preferably be a protein molecule.

The support member may have a functional group capable of reacting with the alkoxyamine group of the sphingosine-1-phosphate analogue. The functional group may be an epoxy group or a carbonyl group, but the present specification is not limited thereto. The carbonyl group may be included in an aldehyde, a ketone, carboxylic acid and derivatives thereof such as an ester and an amide, and a ketene, but the present specification is not limited thereto.

The support member may be in physical or chemical contact with the plate.

The support member may immobilize the S1P analogue directly and/or indirectly. The support member includes a functional group, and the functional group and the alkoxyamine group of the S1P analogue may form a bond through a chemical reaction. Thus, the S1P analogue may be immobilized directly and/or indirectly on a support member containing a functional group. For example, the S1P-alkoxyamine compound can be immobilized directly on the epoxidized support member. Alternatively, for example, the S1P-alkoxyamine compound may be immobilized directly and/or indirectly on a support member containing a carbonyl group.

The support member may contact the plate. The support member may indirectly immobilize the S1P analogue to the plate through contact with the plate.

When the S1P analogue is directly immobilized on the plate or the support member, a separate linker is not necessarily required. That is, it does not matter whether or not there is a linker. Thus, in processes of manufacturing an immunodiagnostic kit by using a S1P analogue, the manufacturing processes may be reasonably simplified compared to conventional manufacturing processes.

The immunodiagnostic kit may be an enzyme-linked immunosorbent assay (ELISA) kit or a point-of-care testing (POCT) kit, but the present specification is not limited thereto. The ELISA includes direct ELISA, indirect ELISA, sandwich ELISA, and competitive ELISA.

### MODE

Hereinafter, the present specification will be described in further detail with reference to examples. These examples are provided for illustrative purposes only, and it will be obvious to those of ordinary skill in the art that the scope of the content disclosed by the present specification should not be construed as being limited by these examples.

The terms used in the examples are merely used to describe specific examples and are not intended to limit the examples. Singular expressions include plural expressions unless the context clearly indicates otherwise. Throughout the present specification, unless the context requires otherwise, the terms “contains” and “includes” should be understood as including stated processes or elements or a group of processes or elements, but not precluding any other processes or elements, or a group of processes or elements.

Various changes can be made to the examples as described below. The present specification is not intended to be limited to the examples described below and should be understood as including all changes, equivalents, or substitutes thereof.

Unless otherwise defined, all terms used herein including technical or scientific terms have the same meaning as those generally understood by those of ordinary skill in the art. It should be understood that terms generally used, which are defined in a dictionary, have the same meaning as in the context of the related art, and the terms are not interpreted with an ideal or excessively formal meaning unless otherwise clearly defined in the present application.

In describing the examples, detailed descriptions of the related art are omitted when it is deemed that they may unnecessarily obscure the essence of the examples.

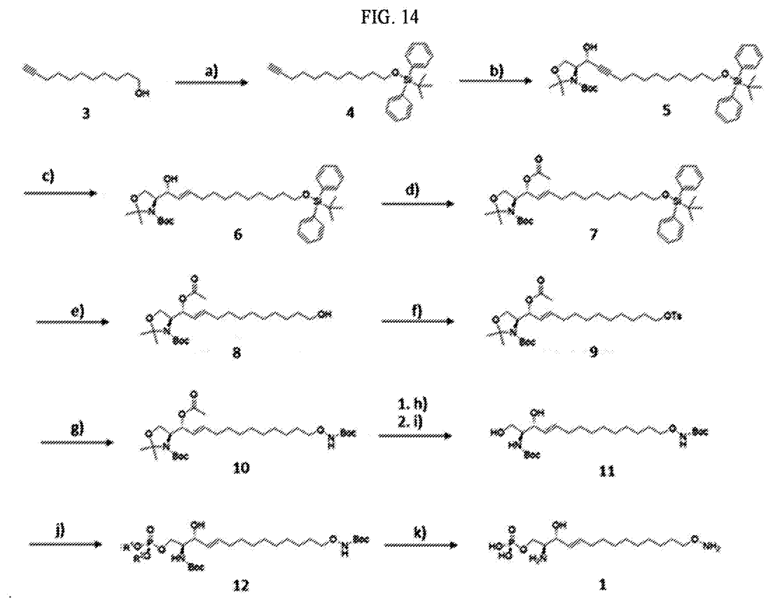

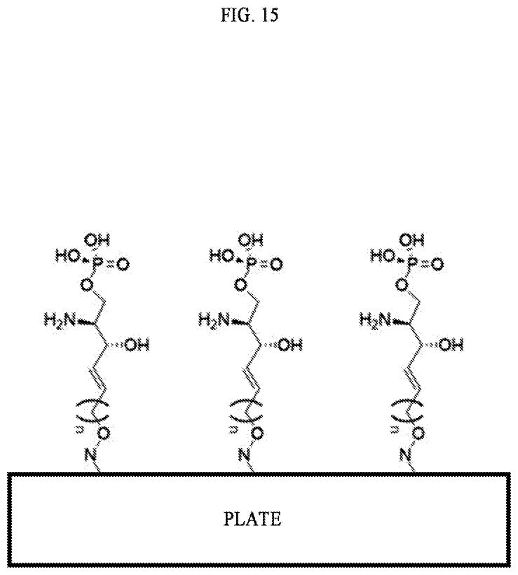

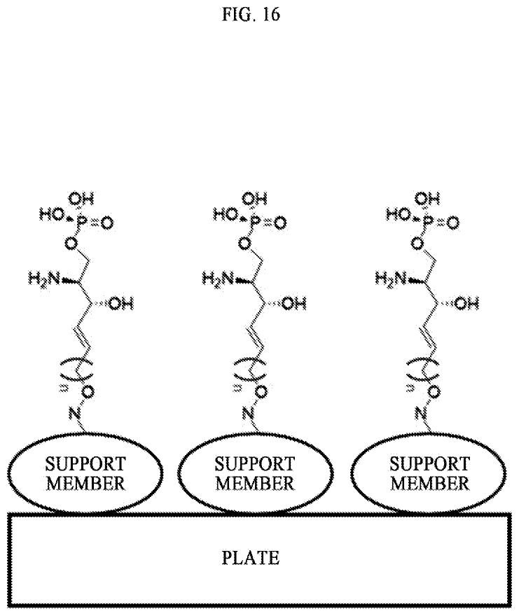

The synthetic approach described in the present examples mainly uses conventional organic chemistry and results in the preparation of a S1P analogue by the continuous addition of structural elements. Respective processes of a synthesis method as described in these examples are illustrated in FIGS. 3 to 13, and the overall process is illustrated in FIG. 14. Compound numbers in the synthesis descriptions below denote numbered structures illustrated in FIGS. 3 to 13.

### [Example 1] Synthesis of Compound 4 from Compound 3

Compound 3 (1 g, 29.7124 mmol) was dissolved in dimethylformamide (DMF) (40 mL) under an Ar atmosphere, and imidazole (3.03 g, 44.5686 mmol) was added thereto. After the temperature of the reaction mixture was lowered to 0° C., tert-butyldiphenylsilyl chloride (TBDPSCl) (9.27 mL, 35.6549 mmol) was slowly added dropwise, and the temperature was raised to room temperature and stirred for 4 hours. After completion of the reaction, the mixture was concentrated under reduced pressure to remove the dimethylformamide, and then the residue was dissolved in dichloromethane (DCM; methylene chloride; MC), washed with an aqueous NaHCO₃ solution and brine, and then dried over Na₂SO₄, followed by filtration and concentration. The concentrate was separated and purified by column chromatography to obtain compound 4 (12 g, 98%) (FIG. 3).

### [Example 2] Synthesis of Compound 5 from Compound 4

Compound 4 (12.2 g, 29.99 mmol) was dissolved in dry tetrahydrofuran (THF) (300 ml) under an Ar atmosphere, and n-butyllithium (22.5 ml, 44.9 mmol; 2M in hexane) was slowly added dropwise at −70° C. The resultant solution was stirred at the same temperature for 30 minutes, and the cooling bath was removed therefrom, followed by stirring for 60 minutes. The temperature of the resultant mixture was lowered to less than −70° C., and Garner's aldehyde (6.88 g, 29.9 mmol) dissolved in dry THF (20 ml) was added dropwise. The resultant solution was stirred at the same temperature for 30 minutes and the cooling bath was removed therefrom. After 2.5 hours, an aqueous NH₄Cl saturated solution was added to terminate the reaction. The reaction product was extracted with DCM (200 ml×2). The organic layer was dried over Na₂SO₄, filtered, and concentrated. The concentrate was separated and purified by column chromatography to obtain compound 5 (9.9 g, 52%) (FIG. 4).

### [Example 3] Synthesis of Compound 6 from Compound 5

Compound 5 (9.9 g, 15.6 mmol) was dissolved in dry THF (100 ml) under an Ar atmosphere, and Red-Al 70% in toluene (13.22 ml, 66.7 mmol, 60% in toluene) was slowly added dropwise at −70° C. After 1 hour, the cooling bath was removed and a reaction was allowed to occur overnight at room temperature. In an ice-water bath, an aqueous NH₄Cl saturated solution (200 ml) was added to terminate the reaction. The resultant solution was extracted with ethyl acetate (EtOAc) (300 ml×3). The organic layer was dried over Na₂SO₄, filtered, and concentrated. The concentrate was separated and purified by column chromatography to obtain compound 6 (4.5 g, 45.3%) (FIG. 5).

### [Example 4] Synthesis of Compound 7 from Compound 6

Compound 6 (1.9 g, 2.98 mmol) was dissolved in pyridine (6 ml) and acetic anhydride (3 mL) was added dropwise. After confirming the completion of a reaction by monitoring with thin layer chromatography (TLC), the reaction product was washed twice with 1 M hydrochloric acid (HCl) and washed once with brine, followed by drying over Na₂SO₄, filtration, and concentration, to obtain compound 7 (2.02 g, 100%). Compound 7 was used in the subsequent reaction without additional purification (FIG. 6).

### [Example 5] Synthesis of Compound 8 from Compound 7

Compound 7 (crude 2.02 g, 2.98 mmol) was dissolved in dry THF (50 ml) and n-Bu₄NF (13.97 ml, 13.97 mmol; 1 M in THF) was slowly added dropwise at 0° C. After confirming the completion of a reaction by TLC monitoring, an aqueous NH₄Cl saturated solution (200 ml) was added to terminate the reaction. The reaction product was extracted with EtOAc (300 ml×3). The extracted organic layer was dried over Na₂SO₄, filtered, and concentrated to obtain compound 8 (1.32 g, 100%). Compound 8 was used in the subsequent reaction without additional purification (FIG. 7).

### [Example 6] Synthesis of Compound 9 from Compound 8

Compound 8 (crude 1.32 g, 2.98 mmol) was dissolved in pyridine (20 ml) and p-toluenesulfonyl chloride (p-TsCl) (9.93 g, 52.1 mmol) was slowly added dropwise at 0° C. After confirming the completion of a reaction by TLC monitoring, the reaction product was washed twice with 1M HCl and washed once with brine, followed by drying over Na₂SO₄, filtration, and concentration. The concentrate was separated and purified by column chromatography to obtain compound 9 (1.57 g, 88.4%) (FIG. 8).

### [Example 7] Synthesis of Compound 10 from Compound 9

Compound 9 (1.57 g, 2.64 mmol) was dissolved in diethyl ether (15 mL), 1,8-Diazabicyclo[5.4.0]undec-7-ene (DBU) (1.967 mL, 13.17 mmol) was added dropwise, and then N-Boc-hydroxylamine (1.754 g, 13.17 mmol) was added thereto. After 15 minutes, the reaction mixture was concentrated under reduced pressure to remove diethyl ether, and then a reaction was allowed to occur overnight at room temperature. After confirming the completion of a reaction by TLC monitoring, the reaction product was washed with EtOAc (100 mL), 0.1 M KHSO₄, 0.1 M NaOH, and brine, and then the organic layer was dried over Na₂SO₄, filtered, and concentrated to obtain compound 10 (1.45 g, 99.1%). Compound 10 was used in the subsequent reaction without additional purification (FIG. 9).

### [Example 8] Synthesis of Compound 2 from Compound 10

Compound 10 (1.45 g, 2.61 mmol) was dissolved in THF (6 mL), and 2N—HCl (6 mL) was added dropwise, followed by heating at 75° C. to cause a reflux reaction. After 2 hours of the reaction, the solvent was completely removed under reduced pressure. The residue was dissolved in THF, and then concentration under reduced pressure was repeated several times to produce compound 2, which is a white solid. The compound was used in the subsequent reaction without additional purification (FIG. 10).

### [Example 9] Synthesis of Compound 11 from Compound 2

Compound 2 was dissolved again in DCM (5 mL) and methanol (MeOH) (5 mL), and tetra-ethylammonium (TEA) (7.3 ml, 52.3 mmol) and Boc₂O (1.71 g, 7.84 mmol) were added thereto. After confirming the completion of a reaction by TLC monitoring, the reaction product was washed twice with 1 M HCl and twice with brine, followed by drying over Na₂SO₄, filtration, and concentration. The concentrate was separated and purified by column chromatography to obtain compound 11 (0.880 g, 71.0%) (FIG. 11).

### [Example 10] Synthesis of Compound 12 from Compound 11

Compound 11 (0.110 g, 0.232 mmol) was dissolved in dry DCM (10 ml) and N-methylimidazole (NMI) (0.056 mL, 0.697 mmol) was slowly added dropwise at 0° C. Subsequently, PO(OMe)₂Cl was slowly added dropwise at 0° C. After confirming the completion of a reaction by TLC monitoring, the reaction product was washed twice with 1 M HCl and twice with brine, followed by drying over Na₂SO₄, filtration, and concentration. The concentrate was separated and purified by column chromatography to obtain compound 12 (0.110 g, 84.3%) (FIG. 12).

### [Example 11] Synthesis of Compound 1 from Compound 12

Compound 12 (0.110 g, 0.196 mmol) was dissolved in dry acetonitrile (MeCN) (10 ml) and Me₃SiBr (0.26 mL, 1.958 mmol) was slowly added dropwise at 0° C. After 40 minutes, the flask was rinsed with MeCN (10 ml). The ice-water bath was removed, followed by stirring at room temperature. After 90 minutes, a sample was collected to confirm whether or not the reaction was completed by NMR. After confirming the completion of the reaction, water (3 ml) was added and concentration under reduced pressure was performed. The residue was dissolved in ether (100 ml) and extracted with water (50 ml×3). The water layer was concentrated under reduced pressure, and then the residue was purified using reverse column chromatography. RP-18 (40-63 μm) LiChroprep® Merck was used (eluent, 50% H₂O in MeOH to 100% MeOH). As a result, 50 mg of compound 1, which is an ivory solid (68.5%), was obtained (FIG. 13).

While embodiments have been described with reference to limited embodiments and drawings, it will be obvious to those of ordinary skill in the art that various changes and modifications can be made from the foregoing description. For example, even when described techniques are performed in an order different from the described method, or are replaced or substituted with other elements or equivalents thereof, suitable results can be achieved. Therefore, other implementations, other embodiments, and equivalents to claims also fall within the scope of the appended claims.

1.-27. (canceled)

28. A method for producing an analogue of sphingosine-1-phosphate (S1P) of Formula 1, 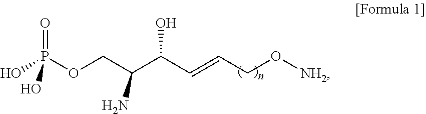
comprising:
a) preparing a compound of Formula 4 by changing a hydroxyl group contained in a compound of Formula 3 into a tert-butyldiphenylsilyl ether group 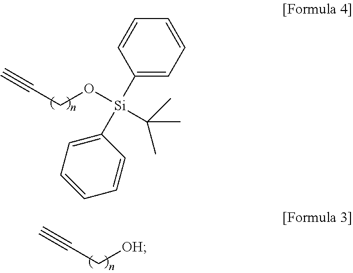
b) preparing a compound of Formula 5 from the compound of Formula 4 by using a Garner's aldehyde of Formula 13 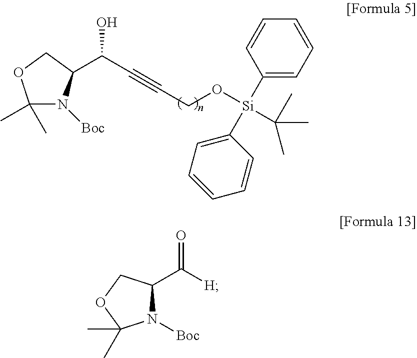
c) preparing a compound of Formula 6 from the compound of Formula 5 by using a reductant 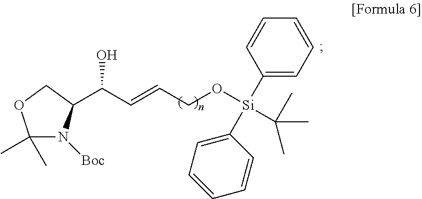
d) preparing a compound of Formula 7 from the compound of Formula 6 by using anhydride 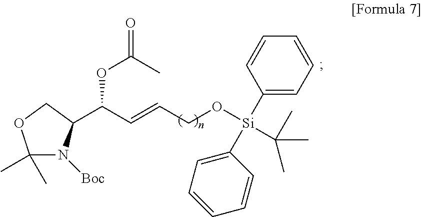
e) preparing a compound of Formula 8 from the compound of Formula 7 by changing the tert-Butyldiphenylsilyl ether group introduced in the a) process into a hydroxyl group 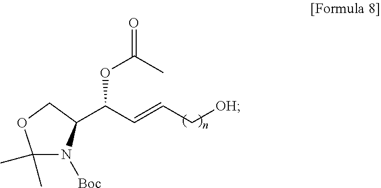
f) preparing a compound of Formula 9 by changing the hydroxyl group contained in the compound of Formula 8 into a p-toluenesulfonate group 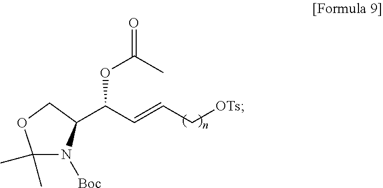
g) preparing a compound of Formula 10 from the compound of Formula 9 by using N-Boc-hydroxylamine of formula 16 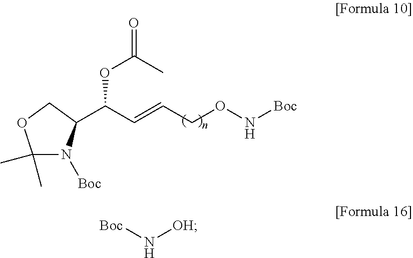
and
h) preparing a compound of Formula 2 from the compound of formula 10 by reacting with tetrahydrofuran (THF) and hydrochloric acid: 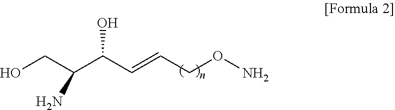
wherein n is selected from an integer of 2 to 13 in the Formula 1 to 10.

29. The method of claim 28, wherein an organolithium compound is used with the Garner's aldehyde in the b) process, wherein the organolithium compound is at least one selected from a group comprising n-butyl lithium, sec-butyl lithium, and tert-butyl lithium.

30. The method of claim 28, wherein the reductant in the c) process is Red-Al (sodium bis(2-methoxyethoxy)aluminumhydride; NaAlH₂(OCH₂CH₂OCH₃)₂) of Formula 14

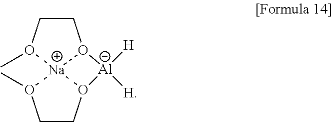

31. The method of claim 28, wherein the anhydride in the d) process is acetic anhydride.

32. The method of claim 28, wherein at least one compound selected from a group comprising KF, CsF, HF, and n-Bu₄NF is used in the process of step e).

33. The method of claim 28, further comprising,
i) preparing a compound of Formula 11 from the compound of Formula 2 by using a di-tert-butyl dicarbonate (Boc₂O), 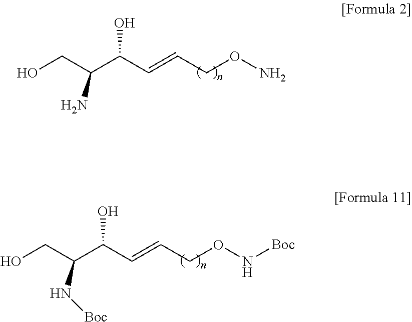
wherein the n is selected from an integer of 2 to 13 in the Formula 2 and 11.

34. The method of claim 33, further comprising,
j) preparing a compound of Formula 12 from the compound of Formula 11 by using a phosphonate ester, 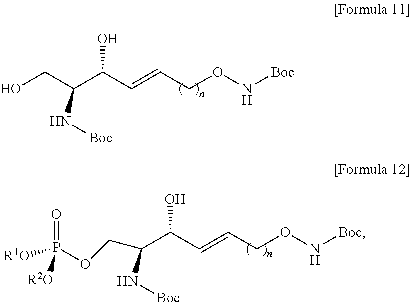
wherein n is selected from an integer from 2 to 13 in the Formula 11 and Formula 12; and wherein R¹ and R² are selected from H and an alkyl group having C1 to C7 in the Formula 12; and wherein the phosphonate ester is at least one selected from a group comprising phosphonate ester of Formula 18, 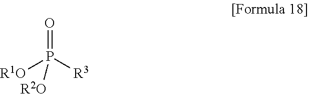
wherein R³ is selected from HSO₄, p-toluenesulfonate, and a halogen atom except for fluorine and R¹ and R² are selected from H and an alkyl group having C1 to C7, in the Formula 18.

35. The method of claim 34, wherein N-methylimidazole (NMI) of a Formula 17 is used with the phosphonate ester,

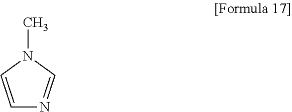

36. The method of claim 34, further comprising,
k) preparing the compound of Formula 1 by changing R¹ and R² contained in the compound of Formula 12 into a hydrogen atom, 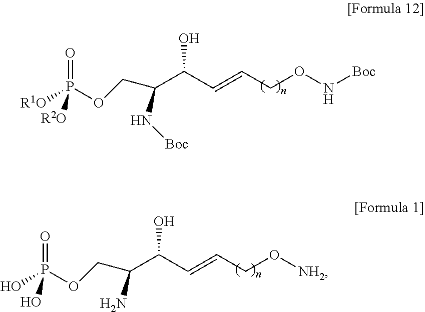
wherein n is selected from an integer from 2 to 13 in the Formula 1 and Formula 12.

37. The method of claim 36, wherein at least one compound selected from a group comprising trimethylsilyl bromide (Me₃SiBr), tribromoborane (BBr₃), hydrobromate (HBr), and dimethylformamide butyllithium is used to change the R¹ and R² into the hydrogen atom.

38. A method for producing an analogue of sphingosine of Formula 2, 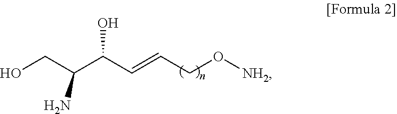
comprising:
a) preparing a compound of Formula 4 by changing a hydroxyl group contained in a compound of Formula 3 into a tert-butyldiphenylsilyl ether group 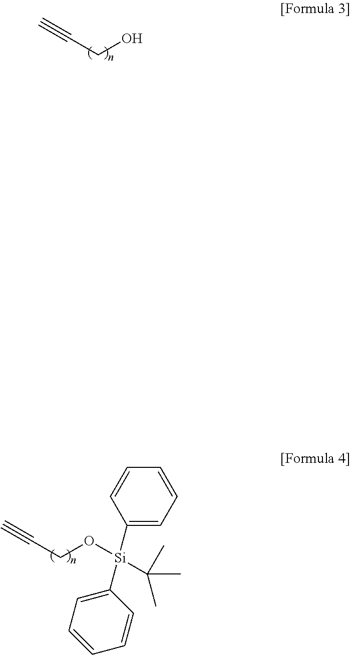
b) preparing a compound of Formula 5 from the compound of Formula 4 by using a Garner's aldehyde of Formula 13 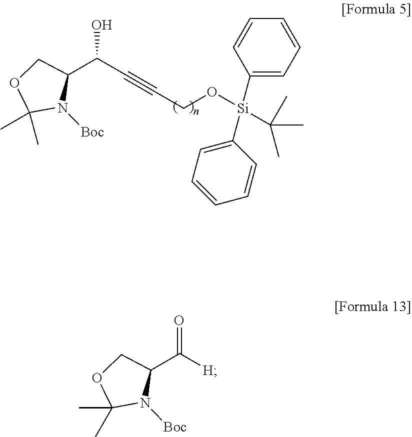
c) preparing a compound of Formula 6 from the compound of Formula 5 by using a reductant 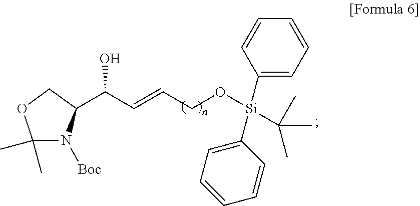
d) preparing a compound of Formula 7 from the compound of formula 6 by using anhydride 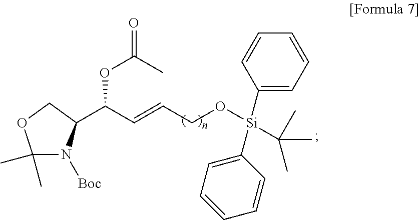
e) preparing a compound of Formula 8 from the compound of Formula 7 by changing the tert-butyldiphenylsilyl ether group introduced in the a) process into a hydroxyl group 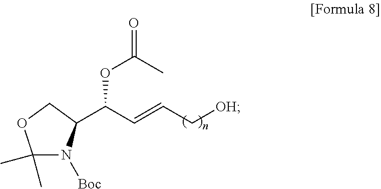
f) preparing a compound of Formula 9 by changing the hydroxyl group contained in the compound of Formula 8 into a p-toluenesulfonate group 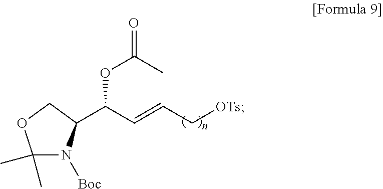
g) preparing a compound of Formula 10 from the compound of Formula 9 by using N-Boc-hydroxylamine of formula 16 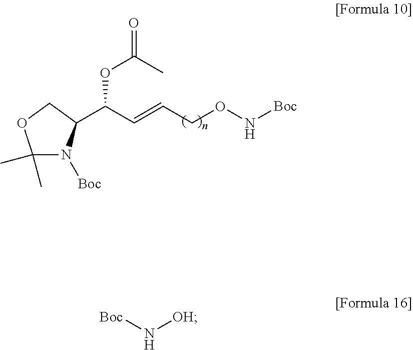
and
h) preparing the compound of Formula 2 from the compound of Formula 10 by reacting with tetrahydrofuran (THF) and hydrochloric acid;
wherein n is selected from an integer of 2 to 13 in the Formula 2 to 20.

39. An analogue of sphingosine-1-phosphate (S1P) is selected from a compound of Formula 1, 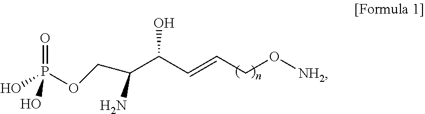
wherein n is selected from an integer of 2 to 13 in the Formula 1.

40. An immunodiagnostic kit comprising:
a plate; and
an analogue of sphingosine-1-phosphate (S1P) selected from a compound of a Formula 1, which is bounded to the plate,
wherein the plate comprises a functional group capable of reacting with an alkoxyamine group in the analogue of sphingosine-1-phosphate (S1P), 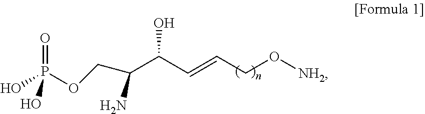
wherein n is selected from an integer of 2 to 13 in the Formula 1.

41. The kit of claim 40, wherein the functional group is selected from an epoxy group and a carbonyl group.

42. The kit of claim 40, wherein the analogue of sphingosine-1-phosphate (S1P) is directly bound to the plate.

43. The kit of claim 40, wherein the immunodiagnostic kit further comprises a support member, wherein the support member comprises a functional group capable of reacting with an alkoxyamine group of the analogue of sphingosine-1-phosphate (S1P).

44. The kit of claim 43, wherein the support member is a protein molecule.

45. The kit of claim 43, wherein the functional group is selected from an epoxy group and carbonyl group.

46. The kit of claim 43, wherein the analogue of sphingosine-1-phosphate (S1P) is directly bound to the support member.

47. The kit of claim 46, wherein immunodiagnostic kit is an enzyme-linked immunosorbent assay (ELISA) kit or a point-of-care testing (POCT) kit.
